(19) United States
(12) Patent Application Publication
(10) Pub. No.: US 2026/0364337 A1
(43) Pub. Date: Nov. 19, 2026

(54) REMOVABLE SHEATH ASSEMBLY

(71) Applicant: Edwards Lifesciences Corporation, Irvine, CA (US)

(72) Inventors: Hien T. Nguyen, Aliso Viejo, CA (US); Michael G. Valdez, Riverside, CA (US)

(73) Assignee: Edwards Lifesciences Corporation, Irvine, CA (US)

(21) Appl. No.: 19/755,591

(22) Filed: Jul. 27, 2026

Related U.S. Application Data
(63) Continuation of application No. 18/532,062, filed on Dec. 7, 2023.
Application No. 18/532,062 is a continuation of application No. PCT/US2022/033192, filed on Jun. 13, 2022.
(60) Provisional application No. 63/212,072, filed on Jun. 17, 2021.

(51) Int. Cl. A61M 60/865; A61M 60/148

(52) U.S. Cl. CPC A61M 60/865; A61M 60/148

(57) ABSTRACT

A sheath assembly can comprise a removable hub. The removable hub can have a lumen extending therethrough and comprise a first circumferential hub portion that spans a first circumferential segment about a longitudinal axis of the removable hub, a second circumferential hub portion that spans a second circumferential segment about the longitudinal axis of the removable hub, and first and second connector portions connecting the first circumferential hub portion and the second circumferential hub portion. The second circumferential segment can be greater than the first circumferential segment. The sheath assembly can comprise a removable shaft configured to extend distally from a distal end of the removable hub, the removable shaft being configured such that separation of the first circumferential hub portion from the second circumferential hub portion causes a first circumferential shaft portion to separate from a second circumferential shaft portion.

### CROSS REFERENCE TO RELATED APPLICATIONS

This application is a continuation of U.S. patent application Ser. No. 18/532,062, filed Dec. 7, 2023, and entitled “REMOVABLE SHEATH ASSEMBLY,” which is a continuation of International Patent Application No. PCT/US2022/033192, filed Jun. 13, 2022, and entitled “REMOVABLE SHEATH ASSEMBLY,” which claims the benefit of U.S. Provisional Patent Application No. 63/212,072, filed Jun. 17, 2021, entitled “REMOVABLE SHEATH ASSEMBLY,” the complete disclosures of which are hereby incorporated by reference herein in its entirety.

### BACKGROUND

The present disclosure generally relates to the field of minimally invasive delivery medical devices and/or therapies. Minimally invasive transcatheter delivery of medical devices and/or therapies to a target site within the body can be utilized to treat any number of conditions. Medical devices and/or therapies can be delivered into the heart to address various heart abnormalities.

### SUMMARY

Described herein are methods and devices relating to a removable sheath assembly configured to allow advancement therethrough of a medical device for minimally invasive transcatheter delivery of the medical device. The removable sheath assembly can comprise a removable hub, a removable shaft, a removable knob and a removable seal, each of the removable hub, removable shaft, removable knob and removable seal can be peeled away and/or lifted away from around the medical device while the medical device is extending through the removable sheath assembly.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages, and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 is a cross-sectional view of a human heart.

FIGS. 2A and 2B are perspective views of an example of a removable sheath assembly in an exploded configuration and an assembled state, respectively. FIG. 2C is a cut-away view of a proximal portion of the removable sheath assembly in the assembled state.

FIGS. 3A and 3B are perspective views of the removable hub of the removable sheath assembly described with reference to FIG. 2 from a distal end and proximal end of the removable hub, respectively. FIG. 3C is a cutaway view of the removable hub. FIGS. 3D and 3E show the removable hub from the distal end and the proximal end of the removable hub, respectively.

FIG. 4A is a perspective view, FIG. 4B is a side view, and FIG. 4C is a view from a proximal end, of the removable knob of the removable sheath assembly described with reference to FIG. 2.

FIG. 5A is a perspective view, and FIG. 5B is a view from a distal end, of the removable seal of the removable sheath assembly described with reference to FIG. 2.

FIGS. 6A, 6B and 6C show an example of a delivery system comprising the removable sheath assembly described with reference to FIG. 2 positioned within an outer sheath.

FIGS. 7A, 7B, and 7C show an example of a process for delivering a medical device to a target site using the removable sheath assembly described with reference to FIG. 2.

FIGS. 8A, 8B, 8C, 8D, and 8E show another example of a process for delivering a medical device to a target site using the removable sheath assembly described with reference to FIG. 2.

FIG. 9 is a flow diagram of an example of a process for deploying a medical device using a removable sheath assembly.

### DETAILED DESCRIPTION

The headings provided herein are for convenience only and do not necessarily affect the scope or meaning of the claimed invention.

The present disclosure provides systems, devices, and methods relating to a removable sheath assembly which can be used to facilitate minimally invasive transcatheter delivery of a medical device to a target site. The removable sheath assembly can comprise a removable hub, a removable shaft, a removable knob, and a removable seal. The removable sheath assembly can be completely removed from around the medical device while a portion of the medical device is at a desired position within a bodily lumen, vessel, chamber and/or cavity.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

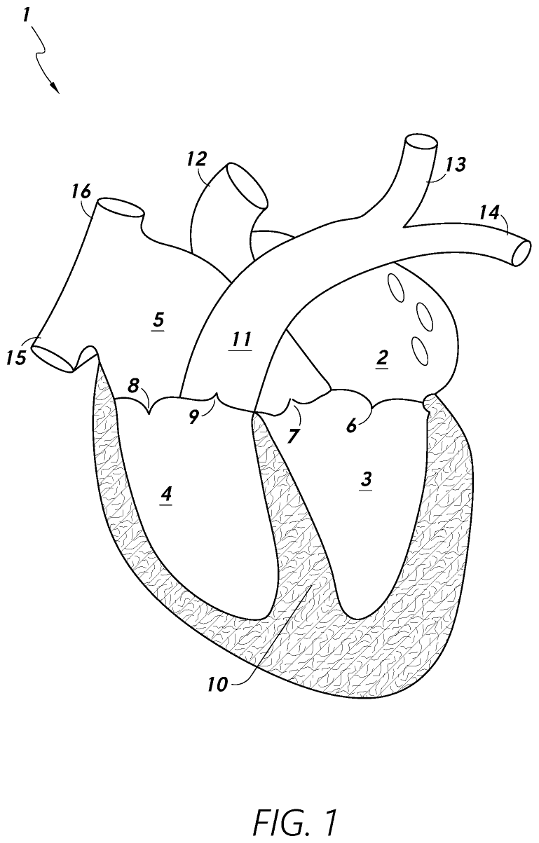

FIG. 1 is a schematic diagram showing various features of a human heart 1. The heart 1 includes four chambers, namely the left atrium 2, the left ventricle 3, the right ventricle 4, and the right atrium 5. A wall of muscle, referred to as the septum 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve 9. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve 9 separates the right ventricle 4 and the pulmonary trunk or artery 11, controlling blood flow therebetween.

In a healthy heart, the heart valves can properly open and close in response to a pressure gradient present during various stages of the cardiac cycle (e.g., relaxation and contraction) to at least partially control the flow of blood to a respective region of the heart and/or to blood vessels. Deoxygenated blood arriving from the rest of the body generally flows into the right side of the heart for transport to the lungs, and oxygenated blood from the lungs generally flows into the left side of the heart for transport to the rest of the body. During ventricular diastole, deoxygenated blood arrive in the right atrium 5 from the inferior vena cava 15 and superior vena cava 16 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary trunk 11 for transport to the lungs (e.g., via the left 14 and right 13 pulmonary arteries), and oxygenated blood can flow from the left ventricle 3 to the aorta 12 for transport to the rest of the body.

A number of conditions can contribute to a higher than normal pressure in the left atrium. Dysfunction of the mitral valve can contribute to elevated left atrial pressure. Conditions such as mitral valve regurgitation and/or stenosis may result in difficulty in pumping blood from the left atrium to the left ventricle, contributing to elevated pressure in the left atrium. Valve stenosis can cause a valve to become narrowed or obstructed. Mitral valve stenosis can restrict blood flow from the left atrium to the left ventricle. Valve regurgitation occurs when a valve does not close properly. For example, regurgitation can occur due to improper coaptation of the valve leaflets, such as due to prolapse of one or more of the valve leaflets. Mitral valve regurgitation can result in blood flow leakage back into the left atrium 2 from the left ventricle 3 when the left ventricle 3 contracts. Restricted flow of blood from the left atrium 2 into the left ventricle 3, and blood flow leakage from the left ventricle 3 back into the left atrium 2 can both contribute to elevated atrial pressure. Dysfunction in the left ventricle 3 can also contribute to elevated left atrial pressure. Elevated left atrial pressure may lead to left atrial enlargement, producing symptoms such as shortness of breath during exertion, fatigue, chest pain, fainting, abnormal heartbeat, and swelling of the legs and feet.

The disclosure herein provides one or more devices and methods related a removable sheath assembly which can be used to facilitate minimally invasive transcatheter delivery of a medical device to a target site. In some instances, the medical device can be used to address and/or ameliorate various heart conditions. The removable sheath assembly can be completely removed after the medical device is at a desired position within a bodily lumen, vessel, chamber and/or cavity, including within a heart. For example, while the medical device is at the desired position, the removable sheath assembly can be removed from around the medical device. The removable sheath assembly can comprise a removable hub, a removable shaft, a removable knob, and a removable seal. Each of the removable hub, removable shaft, removable knob, and removable seal can be removed from around the medical device while the medical device is positioned at the target site. For example, removable hub can comprise a first circumferential hub portion and a second circumferential hub portion configured to be separated from one another for removal of the removable hub. The removable knob can comprise a first circumferential knob portion and a second circumferential knob portion configured to be separated from one another for removal of the removable knob. The first and second circumferential hub portions can span differently sized circumferential segments about a longitudinal axis of the removable hub. The first and second circumferential knob portions can span the same or similarly sized circumferential segments about a longitudinal axis of the removable knob. Separation of the first circumferential hub portion from the second circumferential hub portion can also result separation of a first circumferential shaft portion of the removable shaft from a second circumferential shaft portion of the removable shaft to facilitate removal of the removable shaft. Removing the removable seal can comprise sliding a corresponding portion of the medical device through a slit extending along a length of the removable seal.

The removable sheath assembly can comprise a delivery lumen configured to allow advancement therethrough of at least a portion of the medical device. Each of the removable hub, removable shaft, removable knob, and removable seal can have a respective lumen extending therethrough. For example, the delivery lumen can comprise the lumens of the removable knob, removable seal, and removable shaft. The lumens of the removable knob, removable seal and removable shaft can be aligned with one another to facilitate advancement therethrough of at least a portion of the medical device. The medical device can be advanced through the lumen of the removable knob, then the lumen of the removable seal, and then the lumen of the removable shaft. A distal portion of the removable knob can be received within a proximal portion of the lumen of the removable hub. The removable seal can be received within the lumen of the removable hub distally of the distal portion of the removable knob. In some instances, a proximal portion of the removable shaft can be received within a distal portion of the lumen of the removable hub. The removable shaft can extend distally from a distal end of the removable hub. Distal advancement of the removable knob within the lumen of the removable hub can cause the removable seal to be compressed along a longitudinal dimension, thereby resulting in expansion of the removable seal along one or more lateral dimensions. For example, the removable seal can expand along radial dimensions perpendicular or substantially perpendicular to the longitudinal dimension, causing the removable seal to contact a portion of the medical device extending through the lumen of the removable seal to form a hemostatic seal around the portion of the medical device.

The removable sheath assembly can be used for transcatheter delivery of the medical device to protect the medical device as the medical device is advanced through tortuous anatomical pathways. For example, bends and/or turns along the anatomical pathways can result in damage to the medical device if a removable sheath assembly is not used. In some instances, the removable sheath assembly can provide added mechanical strength to the medical device as the medical device is passed through the anatomical pathways. For example, the medical device alone may not have sufficient mechanical strength to be advanced through bends and/or turns, causing the medical device to become stuck in the pathway and/or damaging the medical device due to bending of the medical device.

In some instances, the removable sheath assembly can be used in combination with an outer sheath in transcatheter delivery of the medical device. The outer sheath can be positioned into the body. A portion of the removable sheath assembly can be subsequently advanced through the delivery lumen of the outer sheath to facilitate navigation of the medical device through the outer sheath. Curves and/or turns in the anatomical pathways can result in bends and/or flattened portions in the outer sheath. Passing portions of the medical device through the outer sheath without a removable sheath assembly as described herein can result in damage to the medical device, including bending of the medical device and damage to one or more portions of an outer surface of the medical device. The removable sheath assembly can provide additional mechanical strength, such as mechanical strength along a longitudinal axis of the medical device, such that the desired portion of the medical device can be successfully advanced through the outer sheath while reducing or eliminating damage to the medical device.

In some instances, the medical device can be delivered into the heart for treatment of various heart conditions. In some instances, the medical device can ameliorate elevated left atrial pressure. Although the medical device is described primarily herein as comprising a percutaneous ventricular assisted device (PVAD), it will be understood that the medical device can be any number of other instruments suited for delivery using the removable sheath assembly. The medical device can be delivered to any number of bodily lumens, vessels, chambers and/or cavities. The medical device can comprise any number of medical implant devices, surgical instruments, and/or medical devices which are temporarily positioned within the body to deliver various therapies.

In some instances, the removable sheath assemblies described herein can be inserted into a femoral artery for delivery of medical devices to the heart. For example, a medical device can be advanced through a removable sheath assembly comprising at least a portion positioned into the femoral artery. The medical device can be advanced into the aorta from the removable sheath assembly and from the aorta into the heart, such as the left heart. It will be understood that the removable sheath assemblies can be inserted into any number of other vessels and/or lumens to facilitate delivery of medical devices to any number of other organs, chambers, vessels and/or lumens. The removable sheath assemblies can be used in any number of different types of minimally invasive transcatheter procedures.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

It will be understood that one or more components of the removable sheath assemblies can undergo various processes in preparation for their use in the procedures, including for example sterilization processes. The removable sheath assemblies can be sterilized removable sheath assemblies. For example, the removable hubs, removable shafts, removable knobs and/or removable seals as described herein can be sterilized removable hubs, sterilized removable shafts, sterilized removable knobs and/or sterilized removable seals.

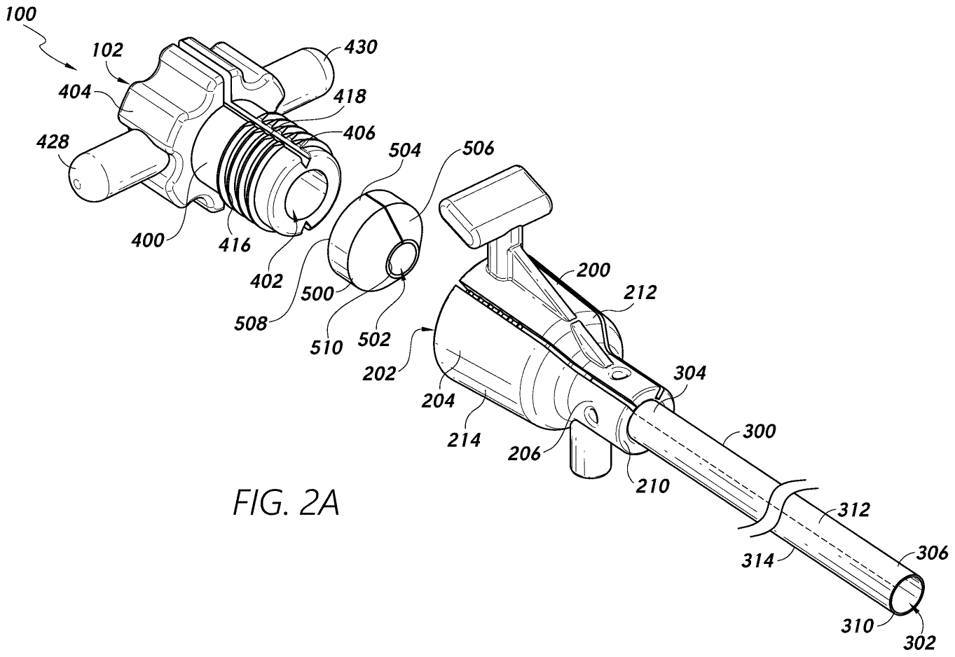

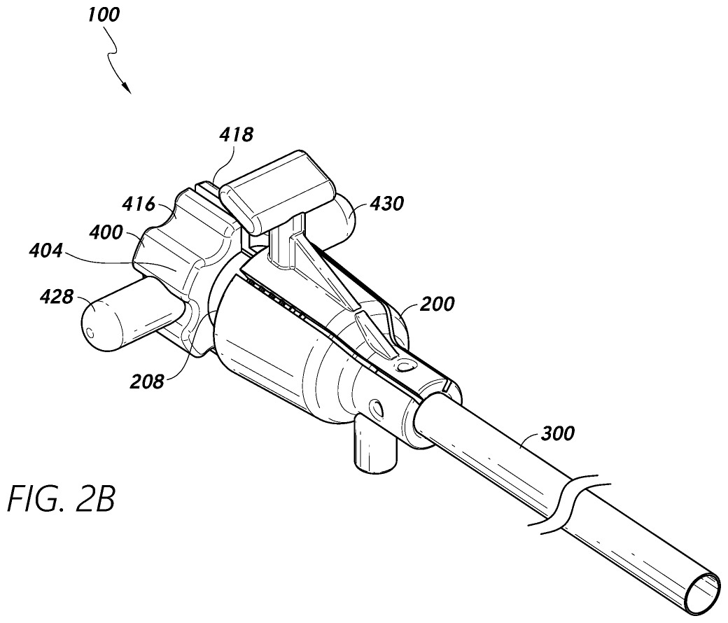

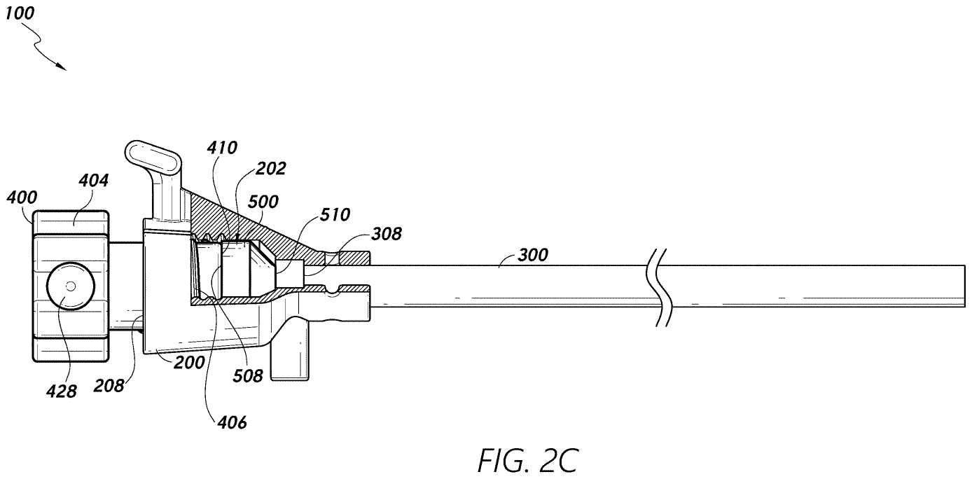

FIGS. 2A, 2B and 2C show an example of a removable sheath assembly 100. FIG. 2A is an exploded perspective view of the removable sheath assembly 100 and FIG. 2B is a perspective view of the removable sheath assembly 100 in an assembled state. FIG. 2C is a cut-away view of a proximal portion of the removable sheath assembly 100 in the assembled state. Referring to FIG. 2A, the removable sheath assembly 100 can comprise a removable hub 200, a removable shaft 300, a removable knob 400 and a removable seal 500. The removable sheath assembly 100 can comprise a delivery lumen 102 configured to allow advancement therethrough of at least a portion of a medical device. Each of the removable hub 200, removable shaft 300, removable knob 400 and removable seal 500 can have a respective lumen extending therethrough. Each of the lumens can be configured to be coaxial with one another while the removable assembly 100 is assembled to provide the delivery lumen 102. Each of the removable hub 200, removable shaft 300, removable knob 400 and removable seal 500 can be removed and/or peeled away from around the medical device after the medical device is desirably positioned.

The removable hub 200 can comprise a lumen 202 extending therethrough. A distal portion of the removable knob 400 can be received in a proximal portion of the lumen 202. The removable seal 500 can be received in the lumen 202 distally of the distal portion of the removable knob 400. The removable shaft 300 can extend distally from a distal end of the removable hub 200. In some instances, the delivery lumen can comprise the lumen of the removable knob 400, the lumen of the removable seal 500 and the lumen of the removable shaft 300. In some instances, the delivery lumen can comprise the lumen of the removable knob 400, the lumen of the removable seal 500, the lumen of the removable shaft 300 and a portion of the lumen 202 of the removable hub 200. For example, at least a portion of the medical device can be advanced through the lumen of the removable knob 400, the lumen of the removable seal 500, and the lumen of the removable shaft 300. In some instances, at least a portion of the medical device can be advanced through the lumen of the removable knob 400, the lumen of the removable seal 500, the lumen 202 of the removable hub 200 and the lumen of the removable shaft 300. In some instances, after the desired portion of the medical device has been advanced through the delivery lumen of the removable assembly 100, each of the removable hub 200, removable shaft 300, removable knob 400 and removable seal 500 can be removed, such as from around the medical device.

The removable hub 200 can comprise a first circumferential hub portion 212 and a second circumferential hub portion 214. The first circumferential hub portion 212 can be configured to be separated from the second circumferential hub portion 214 to facilitate removal of the removable hub 200. The first circumferential hub portion 212 can span a first circumferential segment about a longitudinal axis of the removable hub 200. The second circumferential hub portion 214 can span a second circumferential segment about the longitudinal axis of the removable hub 200. The longitudinal axis of the removable hub 200 can extend between a proximal portion 204 and a distal portion 206 of the removable hub 200, including from a proximal end 208 to a distal end 210 of the removable hub 200. The second circumferential segment can be greater than the first circumferential segment. The first circumferential hub portion 212 and the second circumferential hub portion 214 can be unequally sized such that the removable hub 200 can be separated into two unequally sized portions for removal from around the medical device.

The removable shaft 300 can have an elongate configuration and comprise a lumen 302 extending therethrough. At least a portion of the removable shaft 300 can be configured to be positioned within a bodily lumen, vessel, chamber and/or cavity to facilitate delivery of the medical device. A proximal portion 304 of the removable shaft 300 can be associated with the distal portion 206 of the removable hub 200. The proximal portion 304 of the removable shaft 300 can be coupled to the distal portion 206 of the removable hub 200. In some instances, a proximal end 308 (not shown) of the removable shaft 300 can be received within a distal portion of the lumen 202 of the removable hub 200. The removable shaft 300 can extend distally from the distal end 210 of the removable hub 200 to a distal end 310. The lumen 302 of the removable shaft 300 can be configured to be aligned with the lumen 202 of the removable hub 200 to facilitate advancement therethrough of the medical device. For example, a longitudinal axis of the lumen 302 of the removable shaft 300 can be coaxial with that of the lumen 202 of the removable hub 200. In some instances, the lumen 302 of the removable shaft 300 can extend along a longitudinal axis of the removable shaft 300. The longitudinal axis of the removable shaft 300 can extend between the proximal portion 304 and the distal portion 306 of the removable shaft 300, including from the proximal end 308 to the distal end 310 of the removable shaft 300.

The first circumferential hub portion 212 can be associated with a first circumferential shaft portion 312 of the removable shaft 300. The second circumferential hub portion 214 can be associated with a second circumferential shaft portion 314 of the removable shaft 300. Separation of the first circumferential hub portion 212 from the second circumferential hub portion 214 can be configured to also cause separation of the first circumferential shaft portion 312 from the second circumferential shaft portion 314. For example, the first circumferential hub portion 212 can be coupled to the first circumferential shaft portion 312 such that peeling, lifting away and/or breaking away the first circumferential hub portion 212 also peels, lifts away and/or breaks away the first circumferential shaft portion 312. The second circumferential shaft portion 314 can be larger than the first circumferential shaft portion 312. For example, the first circumferential shaft portion 312 can span a first circumferential segment about a longitudinal axis of the removable shaft 300. The second circumferential shaft portion 314 can span a second circumferential segment about the longitudinal axis of the removable shaft 300. The second circumferential segment can be larger than the first circumferential segment. In some instances, a ratio of the first circumferential segment and the second circumferential segment about the longitudinal axis of the removable shaft 300 can be the same as or similar to a ratio of the first circumferential segment and the second circumferential segment about the longitudinal axis of the removable hub 200.

A distal portion 406 of the removable knob 400 can be configured to be received within the lumen 202 of the removable hub 200. The removable knob 400 can be displaced proximally and distally relative to the removable hub 200 such that the distal portion 406 can be displaced proximally and distally within the lumen 202. The removable seal 500 can be received within the lumen 202 of the removable hub 200 at a position distal of the distal portion 406 of the removable knob 400. Distal displacement of the distal portion 406 within the lumen 202 can be configured to result in compression of the removable seal 500 along its longitudinal axis. The longitudinal axis of the removable seal 500 can extend between a proximal portion 504 and a distal portion 506 of the removable seal 500, including from a proximal end 508 to a distal end 510 of the removable seal 500. Compression of the removable seal 500 along its longitudinal axis can result in expansion of the removable seal 500 along one or more lateral dimensions perpendicular or substantially perpendicular to its longitudinal axis. The removable seal 500 can comprise a lumen 502 extending therethrough. The lumen 502 can be configured to be aligned with the removable knob lumen 402 and the removable shaft lumen 302. For example, a longitudinal axis of the removable seal lumen 502 can be coaxial with that of the removable knob lumen 402 and that of the removable shaft lumen 302. The longitudinal axis of the removable seal lumen 502 can be coaxial with the longitudinal axis of the removable seal 500. During delivery of the medical device, at least a portion of the medical device can be extending through the lumen 502 of the removable seal 500. The expansion of the removable seal 500 along one or more lateral dimensions can result in the removable seal 500 contacting the portion of the medical device extending through the lumen 502 and forming a hemostatic seal around the portion of the medical device. For example, compression of the removable seal 500 along its longitudinal axis can result in expansion along one or more radially extending dimensions that are perpendicular or substantially perpendicular to the longitudinal axis of the removable seal 500.

Referring to FIG. 2B, a perspective view of the removable sheath assembly 100 in an assembled state is shown. In the assembled state, a proximal portion 404 of the removable knob 400 can extend proximally of the removable hub 200. A first handle 428 and a second handle 430 can extend from the proximal portion 404 to facilitate separation of the removable knob 400 into two pieces. The removable knob 400 can comprise a first circumferential knob portion 416 and a second circumferential knob portion 418. The first circumferential knob portion 416 can be configured to be separated from the second circumferential knob portion 418 to facilitate removal of the removable knob 400. The first circumferential knob portion 416 can span a first circumferential segment about a longitudinal axis of the removable knob 400. The second circumferential knob portion 418 can span a second circumferential segment about the longitudinal axis of the removable knob 400. In some instances, the first and second circumferential segments can be equally or substantially equally sized. An operator, such as a surgeon, can manipulate the first handle 428 and the second handle 430 to separate the first circumferential knob portion 416 and the second circumferential knob portion 418 from one another.

FIG. 2C shows a cutaway view of a proximal portion of the assembled removable sheath assembly 100 and a side view of the removable shaft 300. The proximal end 308 of the removable shaft 300 is shown as being received within the lumen 202 of the removable hub 200. In some instances, the proximal end 308 of the removable shaft 300 can be spaced from the distal end 510 of the removable seal 500. FIG. 2C shows the removable seal 500 received within the lumen 202 of the removable hub 200 and positioned distally of the distal portion 406 of the removable knob 400 within the lumen 202. As described in further detail herein, distal advancement of the distal portion 406 of the removable knob 400 relative to the removable hub 200 can result in longitudinal compression of the removable seal 500. Distal advancement of the removable knob 400 can result in distal displacement of the proximal end 508 of the removable seal 500. In some instances, a distal end 410 of the removable knob 400 can be in direct contact with the proximal end 508 of the removable seal 500. A position of the distal end 510 of the removable seal 500 can be fixed relative to the removable hub 200 and/or the removable knob 400 such that distal advancement of the removable knob 400 can result in a compressive force exerted upon the removable seal 500 along its longitudinal axis. The removable seal 500 can expand along one or more lateral dimensions due to compression along its longitudinal axis. Expansion along the one or more lateral dimensions can result in reduction of a diameter of the lumen 502 such that the removable seal 500 can contact a portion of the medical device extending therethrough to provide a seal around the portion of the medical device. For example, longitudinal compression of the removable seal 500 can result in its radial expansion along dimensions perpendicular or substantially perpendicular to the longitudinal axis. Expansion along radially extending dimensions that are perpendicular or substantially perpendicular to the longitudinal axis can result in portions of the removable seal 500 defining the lumen 502 contacting and squeezing the portion of the medical device extending therethrough. Contact between the removable seal 500 and the portion of the medical device can provide a hemostatic seal. Distal advancement of the removable knob 400 can thereby longitudinally compress the removable seal 500 to provide a hemostatic seal around the portion of the medical device extending through the lumen 502 of the removable seal 500. The proximal portion 404 of the removable knob 400 is shown as extending proximally of the proximal end 208 of the removable hub 200. The first handle 428 is shown in the figure. For example, manipulation of the proximal portion 404 of the removable knob 400, including the handles 428, 430 extending from the proximal portion 404, can be performed to distally advance the removable knob 400 relative to the removable hub 200.

The removable sheath assembly 100 can be used for transcatheter delivery of the medical device to protect the medical device as the medical device is advanced through tortuous anatomical pathways. The removable sheath assembly 100 can provide added mechanical strength to the medical device as the medical device is passed through the anatomical pathways. As described in further detail herein, in some instances, the removable sheath assembly 100 can be used in combination with an outer sheath in transcatheter delivery of the medical device. A portion of the removable sheath assembly 100 can be advanced through the delivery lumen of the outer sheath to facilitate navigation of the medical device through the outer sheath. The removable sheath assembly 100 can provide additional mechanical strength, such as mechanical strength along a longitudinal axis of the medical device, such that the desired portion of the medical device can be successfully advanced through the outer sheath while reducing or eliminating damage to the medical device. In some instances, the removable sheath assembly 100 can provide protection for the medical device as the medical device is advanced through a seal stack in the hub of the outer sheath. For example, the removable sheath assembly 100 can prevent a coating on an exterior surface of the medical device from being scraped off as the medical device is advanced through the seal stack.

It will be understood that although the description primarily describes the medical device as being positioned within the delivery lumen 102 of the removable sheath assembly 100, the medical device can be received within a lumen of a delivery catheter while positioned within the delivery lumen 102 of the removable sheath assembly 100. For example, a corresponding portion of the medical device can be received within the lumen extending through a respective portion of the delivery catheter while the respective portion of the delivery catheter is slidably disposed within the delivery lumen 102 of the removable sheath assembly 100.

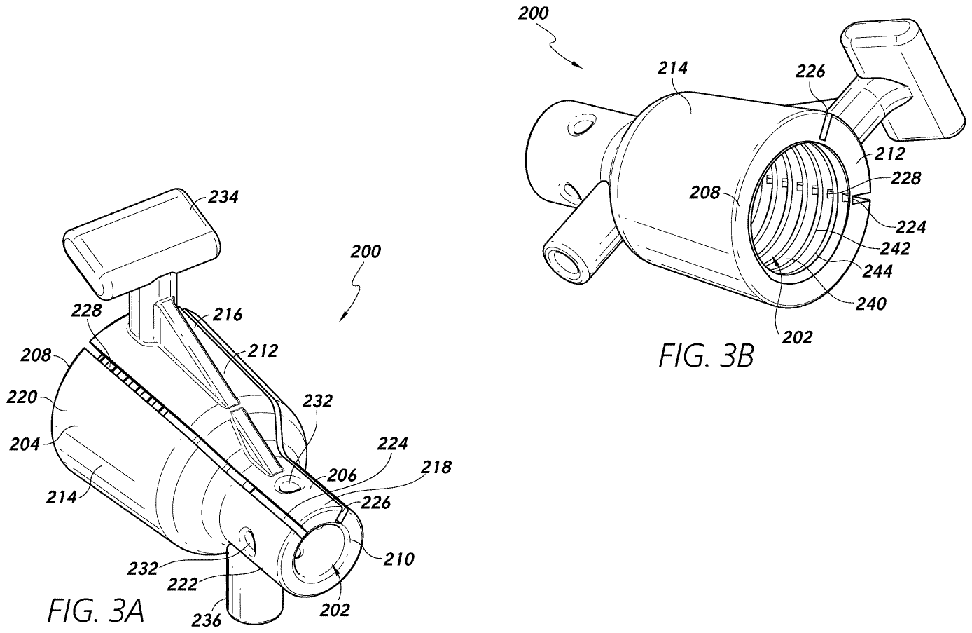

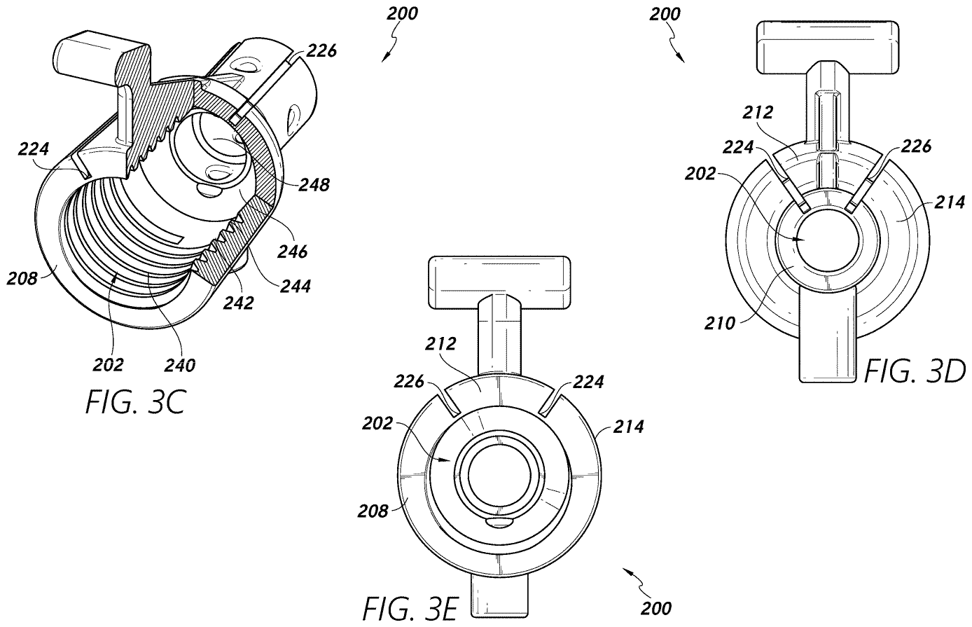

FIGS. 3A through 3E show the removable hub 200 in further detail. FIG. 3A is a perspective view of the removable hub 200 from the distal end 210 of the removable hub 200. FIG. 3B is a perspective view of the removable hub 200 from the proximal end 208 of the removable hub 200. FIG. 3C is a cutaway view of the removable hub 200. FIG. 3D is a view of the removable hub 200 from the distal end 210 of the removable hub 200, and FIG. 3E is a view of the removable hub 200 from the proximal end 208.

Referring to FIG. 3A, the removable hub 200 can comprise a first connector portion 224 and a second connector portion 226 extending between and connecting the first circumferential hub portion 212 and the second circumferential hub portion 214. Each of the first and second connector portions 224, 226 can extend along a length of the removable hub 200. For example, the first and second connector portions 224, 226 can extend along an entire or substantially an entire length of the removable hub 200. The first circumferential hub portion 212 and the second circumferential hub portion 214 can be configured to be separated from one another along the first and second connector portions 224, 226. The first circumferential hub portion 212, the first connector portion 224, the second circumferential hub portion 214, and the second connector portion 226 together span circumferentially around the longitudinal axis of the removable hub 200 such that breaking the removable hub 200 along the first and second connector portions 224, 226 separates the removable hub 200 into two pieces.

The removable hub 200 can comprise a handle 234 extending from a proximal portion 216 of the first circumferential hub portion 212 to allow manipulation of the first circumferential hub portion 212 in order to separate the first circumferential hub portion 212 from the second circumferential hub portion 214. In some instances, the handle 234 can comprise at least a portion that extends laterally from the proximal portion 216, for example including a portion which is perpendicular or substantially perpendicular relative to the longitudinal axis of the removable hub 200. In some instances, pivoting the handle 234 distally can separate the first circumferential hub portion 212 from the second circumferential hub portion 214 along the first and second connector portions 224, 226. An operator, such as a surgeon, can advantageously peel away the first circumferential hub portion 212 using one hand, allowing a free hand for other tasks. For example, pivoting the handle 234 distally can initiate separation of the proximal portion 216 of the first circumferential hub portion 212 from a proximal portion 220 of the second circumferential hub portion 214. The operator can continue to pull on the handle 234 to lift and/or peel the first circumferential hub portion 212 away from the second circumferential hub portion 214.

As described herein, the first circumferential shaft portion 312 of the removable shaft 300 can be associated with, such as coupled to, the first circumferential hub portion 212. The second circumferential shaft portion 314 of the removable shaft 300 can be associated with, such as coupled to, the second circumferential hub portion 214. Lifting away the first circumferential hub portion 212 can separate the first circumferential shaft portion 312 from the second circumferential shaft portion 314. The proximal portion 304 of the removable shaft 300 can be coupled to the removable hub 200 in any one of a number of different manners. In some instances, the proximal portion 304 of the removable shaft 300 can be received within a distal portion of the lumen 202 of the removable hub 200. The proximal portion 304 of the shaft 300 can be coupled to the removable hub 200 within the distal portion of the lumen 202. In some instances, the distal portion 206 of the removable hub 200 can comprise a plurality of openings 232 configured to allow placement of adhesive therethrough so as to bond the proximal portion 304 of the shaft 300 to the removable hub 200. For example, the distal portion 218 of the first circumferential hub portion 212 and the distal portion 222 of the second circumferential hub portion 214 can each comprise one or more openings 232 extending therethrough to allow placement of adhesive for bonding to the first circumferential shaft portion 312 and the second circumferential shaft portion 314, respectively. In some instances, the distal portion 206 of the removable hub 200 can comprise a flush port 236 configured to allow infusion of fluid for flushing the lumen 202 of the removable hub 200. In some instances, the flush port 236 can be proximal of the one or more openings 232. In some instances, the removable shaft 300 can be coupled to the removable hub 200 using an overmolding process. As described herein, in some instances, the proximal end 308 of the removable shaft 300 can be spaced from the distal end 510 of the removable seal 500. In some instances, the proximal end 308 of the removable shaft 300 can be at a position within the lumen 202 distal of where the flush port 236 is in fluid communication with the lumen 202 to allow desired flushing of the lumen 202.

Referring again to FIG. 3A, the first and second connector portions 224, 226 can each comprise a plurality of openings. The plurality of openings extending through a thickness of the first and second connector portions 224,226 can provide breaks in the connection between the first circumferential hub portion 212 and the second circumferential hub portion 214, thereby facilitating separating the first circumferential hub portion 212 and the second circumferential hub portion 214 from one another. As shown in FIG. 3A, at least a portion of the first connector portion 224, such as a proximal portion, can comprise a first plurality of openings 228 spaced from one another along a longitudinal dimension. In some instances, the first plurality of openings 228 can be only on the proximal portion of the first connector portion 224. For example, the first plurality of openings 228 can be on the proximal portion of the first connector portion 224 at regular intervals along the longitudinal dimension. The second connector portion 226 can comprise a second plurality of openings 230 (not shown) on at least a portion, such as a proximal portion. In some instances, the second plurality of openings 230 can be on the proximal portion of the second connector portion 226 at regular intervals along a longitudinal dimension of the second connector portion 226. In some instances, the first and second plurality of openings 228, 230 can be at positions along the respective connector portion 224, 226 which intersect with troughs of a plurality of grooves 244 on an inner wall surface 240 of the removable hub 200. Crests of the plurality of grooves 244 can be the space between adjacent openings of the first and second plurality of openings 228, 230. The first and second plurality of openings 228, 230 can facilitate initiating the separation of the first circumferential hub portion 212 and the second circumferential hub portion 214. In some instances, connector portions 224, 226 comprising the plurality of openings 228, 230 along a portion thereof can provide desired mechanical strength for holding together adjacent portions of the removable hub 200 while facilitating subsequent separation of the adjacent portions, such as the first and second circumferential hub portions 212, 214.

Referring to FIG. 3B, the inner wall surface 240 of the removable hub 200 can define the lumen 202. A proximal portion 242 of the inner wall surface 240 can comprise the plurality of grooves 244 configured to mate with a plurality of grooves on an exterior surface of the removable knob 400. As described in further detail herein, the distal portion 406 of the removable knob 400 can comprise the plurality of grooves on the exterior surface to mate with the plurality of grooves 244 on the inner wall surface 240 while the distal portion 406 of the removable knob 400 is positioned within the lumen 202. Mating between the grooves of the removable hub 200 and removable knob 400 can facilitate controlled distal and/or proximal displacement of and relative longitudinal positioning of the removable knob 400 relative to the removable hub 200. In some instances, the plurality of grooves 244 of the removable hub 200 can comprise threads configured to mate with corresponding threads on the removable knob 400. The threads can allow relative rotation of the removable knob 400 and the removable hub 200 such that a continuous change in their relative longitudinal position can be achieved. For example, the removable knob 400 can be rotated around its longitudinal axis in a first direction relative to the removable hub 200 to advance the distal portion 406 of the removable knob 400 distally within the lumen 202 of the removable hub 200 such that their relative longitudinal position can change along a continuous gradient. The distance advanced can be determined based on the extent of longitudinal compression achieved in the removable seal 500, such as to achieve the desired hemostatic seal. The removable knob 400 can be rotated around its longitudinal axis in a second opposing direction to withdraw the distal portion 406 proximally within the lumen 202. In some instances, the plurality of grooves 244 can comprise a plurality of parallel or substantially parallel rings. For example, the removable knob 400 can be pushed or pulled relative to the removable hub 200 to distally advance or proximally withdrawal the removable knob 400, respectively, the plurality of grooves 244 serving to hold the removable knob 400 and removable hub 200 at fixed positions relative to one after incremental changes in their relative longitudinal position. Alternatively or in combination, the proximal portion 242 of the inner wall surface 240 can comprise other features, such as protrusions and/or indentations, to facilitate controlled advancement and/or withdrawal of and relative longitudinal positioning of the removable knob 400 and the removable hub 200 within the lumen 202.

The first plurality of openings 228 of the first connector portion 224 is shown. As shown, the first plurality of openings 228 are at positions which coincide with troughs in the plurality of grooves 244 on the inner wall surface 240.

FIG. 3C is a cutaway view from the distal end 210 of the removable hub 200. The lumen 202 of the removable hub 200 is shown. The proximal portion of the lumen 202 can have a diameter larger than the distal portion of the lumen 202. For example, the proximal portion of the lumen 202 can be sized to mate with the distal portion 406 of the removable knob 400. In some instances, the distal portion of the lumen 202 can be sized to receive the proximal portion 304 of the removable shaft 300 and facilitate coupling of the proximal portion 304 of the removable shaft 300 to the removable hub 200 within the lumen 202. In some instances, the proximal portion 304 can have a frictional fit with the removable hub 200 within the distal portion of the lumen 202. For example, a distal portion 248 of the inner wall surface 240 can be configured to engage with the proximal portion 304 of the removable shaft 300, including be in frictional contact with the proximal portion 304 of the removable shaft 300.

As described herein, the removable seal 500 can be configured to be received within the lumen 202 distal of the removable knob 400. The lumen 202 can comprise a portion shaped to receive the removable seal 500. For example, a portion of the lumen 202 distal of the plurality of grooves 244 and proximal of the removable shaft receiving portion can be shaped to receive the removable seal 500. The inner wall surface 240 defining the lumen 202 can comprise an inward taper configured to engage with a corresponding taper of the removable seal 500. For example, a distal portion 506 of the removable seal 500 can have a tapered configuration. The distal end 510 can have a smaller diameter than more proximal portions of the removable seal 500. The distal portion 506 of the removable seal 500 can assume a frustoconical shape. In some instances, the removable seal receiving portion of the lumen 202 can have a frustoconical shape. The removable seal engaging portion 246 of the inner wall surface 240 defining portion of the lumen 202 configured to engage with the distal portion 506 of the removable seal 500 can taper inward toward the longitudinal axis of the removable hub 200, for example forming a frustoconically shaped lumen portion.

FIG. 3D is a view of the removable hub 200 from the distal end 210 of the removable hub 200, and FIG. 3E is a view of the removable hub 200 from the proximal end 208 of the removable hub 200. The first and second connector portions 224, 226 are shown as extending along the entire length of the removable hub 200. The first and second connector portions 224, 226 can comprise at least a portion that has a reduced thickness relative to adjacent portions of the first and second circumferential hub portions 212, 214. The reduced thickness can, for example, facilitate, breaking of the connector portions 224, 226 to facilitate separation of the first and second circumferential hub portions 212, 214 from one another. For example, the first and second connector portions 224, 226 can each comprise portions extending between respective adjacent portions of the first and second circumferential hub portions 212, 214, where the first and second connector portions 224, 226 have a thickness thinner than that of the respective adjacent portions of the first and second circumferential hub portions 212, 214.

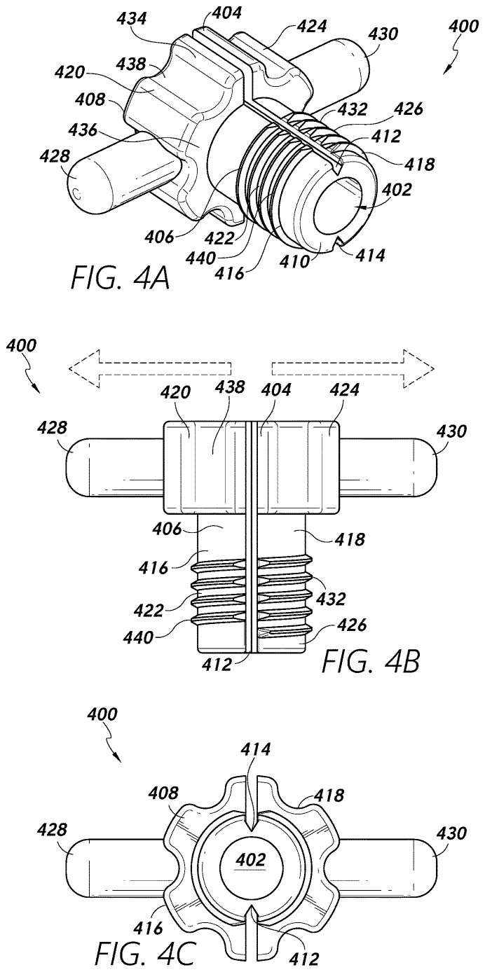

FIGS. 4A through 4C show the removable knob 400 in further detail. FIG. 4A is a perspective view of the removable knob 400. FIG. 4B is a side view and FIG. 4C is a view from a proximal end of the removable knob 400. Referring to FIG. 4A, as described herein, the removable knob 400 can be configured to be separated into a first circumferential knob portion 416 and a second circumferential knob portion 418 to facilitate its removal. The first circumferential knob portion 416 can span a first circumferential segment around a longitudinal axis of the removable knob 400. The second circumferential knob portion 418 can span a second circumferential segment around the longitudinal axis of the removable knob 400. The longitudinal axis can extend between the proximal portion 404 and distal portion 406 of the removable knob 400, such as from a proximal end 408 to the distal end 410 of the removable knob 400. The first and second circumferential segments can be the same or similarly sized. In some instances, the first circumferential knob portion 416 and the second circumferential knob portion 418 can be the same or similar. In some instances, the removable knob 400 can be split into two halves.

The removable knob 400 can comprise a lumen 402 extending therethrough. In some instances, the lumen 402 can extend along a longitudinal axis of the removable knob 400. A longitudinal axis of the lumen 402 can be configured to be coaxial with that of the lumen 302 of the removable shaft 300, the lumen 502 of the removable seal 500 to facilitate advancement therethrough of at least a portion of a medical device.

The removable knob 400 can comprise a first connector portion 412 and a second connector portion 414 configured to couple the first circumferential knob portion 416 and the second circumferential knob portion 418 to one another. For example, the first circumferential knob portion 416, the first connector portion 412, the second circumferential knob portion 418 and the second connector portion 414 together span circumferentially around the longitudinal axis of the removable knob 400. In some instances, the first connector portion 412 and the second connector portion 414 can extend along an entire or substantially entire length of the removable knob 400. In some instances, the first and second connector portions 412, 414 can extend along opposing portions of the removable knob 400. The first and second connector portions 412, 414 can be opposingly positioned such that separating the first circumferential knob portion 416 and the second circumferential knob portion 418 along the first and second connector portions 412, 414 can result in separating the removable knob 400 into two circumferential portions spanning the same or similar circumferential segment around the longitudinal axis. In some instances, the first and second circumferential knob portions 416, 418 are similar or identical portions.

The first and second connector portions 412, 414 are shown as extending along the entire length of the removable knob 400. The first and second connector portions 412, 414 can each comprise at least a portion that has a reduced thickness relative to adjacent portions of the first and second circumferential knob portions 416, 418. The reduced thickness can, for example, facilitate, breaking of the connector portions 412, 414 to facilitate separation of the first and second circumferential knob portions 416, 418 from one another. For example, the first and second connector portions 412, 414 can each comprise portions extending between respective adjacent portions of the first and second circumferential knob portions 416, 418. The first and second connector portions 412, 414 can each have a thickness thinner than that of the respective adjacent portions of the first and second circumferential knob portions 416, 418.

As described herein, the distal portion 406 of the removable knob 400 can be configured to be received within a proximal portion of the lumen 202 of the removable hub 200. An exterior surface 440 of the distal portion 406 can comprise thereon a plurality of grooves 432 configured to mate with the corresponding grooves 244 on the inner wall surface 240 of the removable hub 200. In some instances, the plurality of grooves 432 can comprise threads configured to mate with corresponding threads of the removable hub 200. In some instances, the plurality of grooves 432 can comprise a plurality of parallel or substantially parallel rings configured to mate with corresponding rings on the inner wall surface 240 of the removable hub 200. Alternatively or in combination, the exterior surface 440 of the removable knob 400 can comprise one or more other features, including protrusions and/or indentations, configured to mate with corresponding features on the inner wall surface 240 of the removable hub 200 to allow controlled distal and proximal displacement and longitudinal positioning of the removable knob 400 relative to the removable hub 200.

The removable knob 400 can comprise a first handle 428 and a second handle 430 extending from the proximal portion 404 of the removable knob 400. For example, the first handle 428 and the second handle 430 can extend laterally from the first circumferential knob portion 416 and the second circumferentially knob portion 418. The first handle 428 and the second handle 430 can each comprise at least a portion extending perpendicularly or substantially perpendicularly from the proximal portion 404 of the removable knob 400. The first and second handles 428, 430 can comprise portions comprising opposing orientations relative to one another. In some instances, the first handle 428 can extend laterally from a position on a proximal portion 420 of the first circumferential knob portion 416 and the second handle 430 can extend from a position on a proximal portion 424 of the second circumferential knob portion 418 opposite that of the first handle 428. Pivoting the first and second handles 428, 430 distally can be configured to effect splitting of the removable knob 400 into two portions, for example into two halves. For example, pivoting the first and second handles 428, 430 distally can initiate separation of the proximal portions 420, 424 of the first and second circumferential knob portions 416, 418 from one another, and then continued separation of the distal portions 422, 426 of the first and second circumferential knob portions 416, 418 from one another.

The proximal portion 404 of the removable knob 400 can comprise a circumferential lip 434 protruding laterally beyond the distal portion 406. The circumferential lip 434 can be configured to extend proximally from the proximal end of the removable hub 200 while the distal portion 406 of the removable knob 400 is positioned within the lumen 202 of the removable hub 200. In some instances, a distal end 436 of the circumferential lip 434 can rest against the proximal end of the removable hub 200 to prevent further distal advancement of the removable knob 400 relative to the removable hub 200. In some instances, the first handle 428 and the second handle 430 can extend from the circumferential lip 434. In some instances, the circumferential lip 434 can comprise a plurality of indentations 438 on at least a portion configured to facilitate gripping of the removable knob 400, such as by an operator, to turn the removable knob 400 relative to the removable hub 200.

FIG. 4B is a side view of the removable knob 400. The first and second handles 428, 430 can extend from opposing positions on the proximal portion 404 of the removable knob 400, including from the circumferential lip 434. In some instances, the first and second handles 428, 430 can extend along a dimension perpendicular or substantially perpendicular to the longitudinal axis of the removable knob 400. The connector portion 412 is shown extending along the entire length of the removable knob 400. The plurality of grooves 432 around the distal portion 406 on the exterior surface 440 is shown. The arrows show the directions in which the first circumferential knob portion 416 and the second circumferential knob portion 418 can be separated from one another. Pivoting the first and second handles 428, 430 distally can initiate separation of the proximal portions 420, 424 of the first and second circumferential knob portions 416, 418 from one another. Continued pivoting of the handles 428, 430 can cause separation of the distal portions 422, 426 of the first and second circumferential knob portions 416, 418 from one another.

FIG. 4C is a plan view from the proximal end of the removable knob 400. The first and second connector portions 412, 414 can extend along opposing portions of the removable knob 400 such that the first and second circumferential knob portions 416, 418 span along equally or substantially equally sized circumferential segments around the longitudinal axis of the removable knob 400. The first and second handles 428, 430 can each extend from a position midway between the first and second connector portions 412, 414. In some instances, the first and second handles 428, 430 can extend perpendicularly or substantially perpendicularly relative to the longitudinal axis of the removable knob 400.

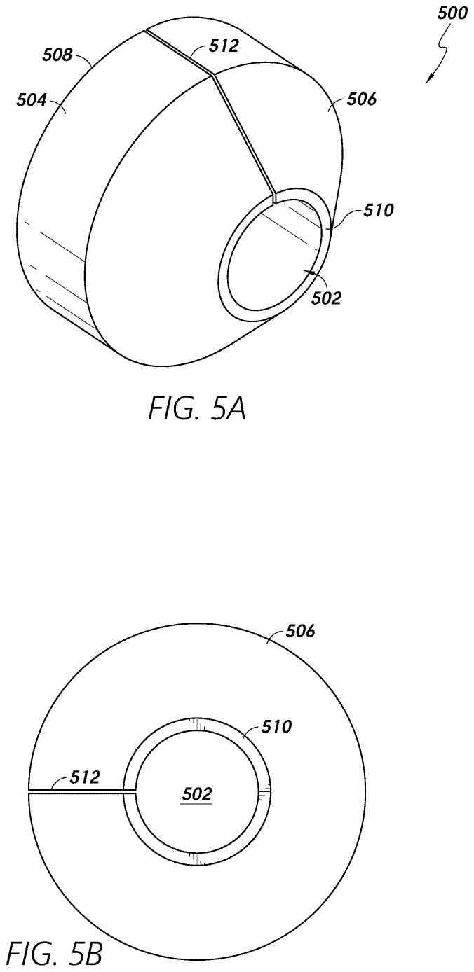

FIG. 5A is a perspective view of the removable seal 500 and FIG. 5B is a view of the removable seal 500 from a distal end 510 of the removable seal 500. The removable seal 500 can comprise a lumen 502 extending therethrough. To facilitate delivery of a medical device using the removable sheath assembly 100, the lumen 502 of the removable seal 500 can be configured to be aligned with the lumen 302 of the removable shaft 300 and the lumen 402 of the removable knob 400 while the removable seal 500 is received within the lumen 202 of the removable hub 200. For example, a longitudinal axis of the lumen 502 can be coaxial with that of the lumen 302 of the removable shaft 300 and the lumen 402 of the removable knob 400. At least a portion of a medical device can be advanced through a delivery lumen of the removable sheath assembly 100 to facilitate delivery of the medical device to a target site within the patient. The delivery lumen can comprise the lumen 502 of the removable seal 500, the lumen 302 of the removable shaft 300 and the lumen 402 of the removable knob 400. In some instances, the lumen 502 can extend along a longitudinal axis of the removable seal 500. The longitudinal axis of the removable seal 500 can extend between a proximal portion 504 and a distal portion 506 of the removable seal 500.

The removable seal 500 can be configured to be received within the lumen 202 of the removable hub 200 and be positioned distally of the portion of the removable knob 400 received within the removable hub lumen 202. The removable seal 500 can be compressible. A position of a distal end 510 of the removable seal 500 within the removable hub lumen 202 can be fixed such that distally advancing the removable knob 400 within the removable hub lumen 202 can result in compression of the removable seal 500 along its longitudinal axis. In some instances, the distal end 410 of the removable knob 400 can be configured to contact a proximal end 508 of the removable seal 500 such that distal advancement of the removable knob 400 can result in the removable knob 400 directly pressing against the proximal end 508 of the removable seal 500 to effect longitudinal compression of the removable seal 500. Lateral expansion of the removable seal can occur due to longitudinal compression of the removable seal 500. The removable seal 500 can expand along one or more lateral dimensions due to longitudinally compressing the removable seal 500. The one or more lateral dimensions can extend perpendicularly or substantially perpendicularly relative to the longitudinal axis of the removable seal 500. Lateral expansion of the removable seal 500 can cause the lumen 502 of the removable seal 500 to reduce in size, for example, reducing a diameter of the lumen 502. The removable seal 500 can thereby contact a portion of the medical device extending through the lumen 502 of the removable seal 500 such that a hemostatic seal can be formed around the portion of the removable seal 500. In some instances, the removable seal 500 can be configured to accommodate a wide range of medical devices. For example, the portion of the medical device extending through the lumen 502 of the removable seal 500 can have a wide variety of widths. In some instances, a diameter of the lumen 502 can be reduced to zero as a result of longitudinal compression of the removable seal 500. In some instances, a material of the removable seal 500 can be selected based in part on a desired compressibility of the removable seal 500 to achieve the target reduction in diameter.

In some instances, a proximal portion 504 of the removable seal 500 can form a cylindrical or substantially cylindrical shape. In some instances, a distal portion 506 of the removable seal 500 can have a tapered configuration, for example the distal end 510 having a smaller diameter than more proximal portions of the removable seal 500. A cross-sectional size of the distal portion 506 can decrease toward the distal end 510. In some instances, the distal portion 506 of the removable seal 500 can assume a frustoconical shape.

As described herein, the lumen 202 of the removable hub 200 can comprise a portion shaped to receive the removable seal 500. The inner wall surface 240 of the removable hub 200 defining the lumen 202 can form a taper that is configured to engage with the distal portion 506 of the removable seal 500. The inner wall surface 240 can comprise a portion which tapers inward, such as toward a longitudinal axis of the lumen 202. For example, the inner wall surface 240 can taper inward to form a frustoconically shaped lumen portion. In some instances, the inner wall surface 240 of the removable hub 200 can form a cylindrically shaped lumen portion that is configured to receive the cylindrically shaped proximal portion 504 of the removable seal 500, and a frustoconically shaped lumen portion distal of the cylindrically shaped lumen portion configured to receive the frustoconically shaped distal portion 506 of the removable seal 500. The removable seal 500 can have a shape configured to facilitate formation of the hemostatic seal around the portion of the medical device extending through its lumen 502. For example, the tapered portion of the inner wall surface 240, such as the portion forming the frustoconically shaped lumen, can be configured to contact the tapered distal portion 506 of the removable seal 500. Distal advancement of the distal portion 406 of the removable knob 400 relative to the removable hub 200 can result in pressing the distal portion 506 of the removable seal 500 against a corresponding portion of the inner wall surface 240 of the removable hub 200. Advancing the removable knob 400 distally within the lumen 202 of the removable hub 200 is configured to push the distal portion 506 of the removable seal 500 against the inner wall surface portion forming the frustoconical shape. The taper of the inner wall surface portion of the removable hub 200 can facilitate forcing portions of the removable seal 500 to engage with the portion of the medical device extending within the lumen 502, as the removable seal 500 expands laterally. As described herein, a position of the distal end 510 of the removable seal 500 can be fixed relative to the removable hub 200 and/or the removable knob 400. In some instances, contact between the inner wall surface 240 defining the frustoconically shaped lumen portion and the frustoconically shaped distal portion 506 of the removable seal 500 can facilitate maintaining the position of the distal end 510 of the removable seal 500.

In some instances, the removable seal 500 can comprise a slit 512 extending along an entire length thereof to allow removal of the removable seal 500 from around the medical device. The removable seal 500 can be circumferentially discontinuous such that it comprises a gap extending along a longitudinal dimension. For example, a portion of the medical device extending through the lumen 502 of the removable seal 500 can be passed through the slit 512 to peel the removable seal 500 from around the portion of the medical device.

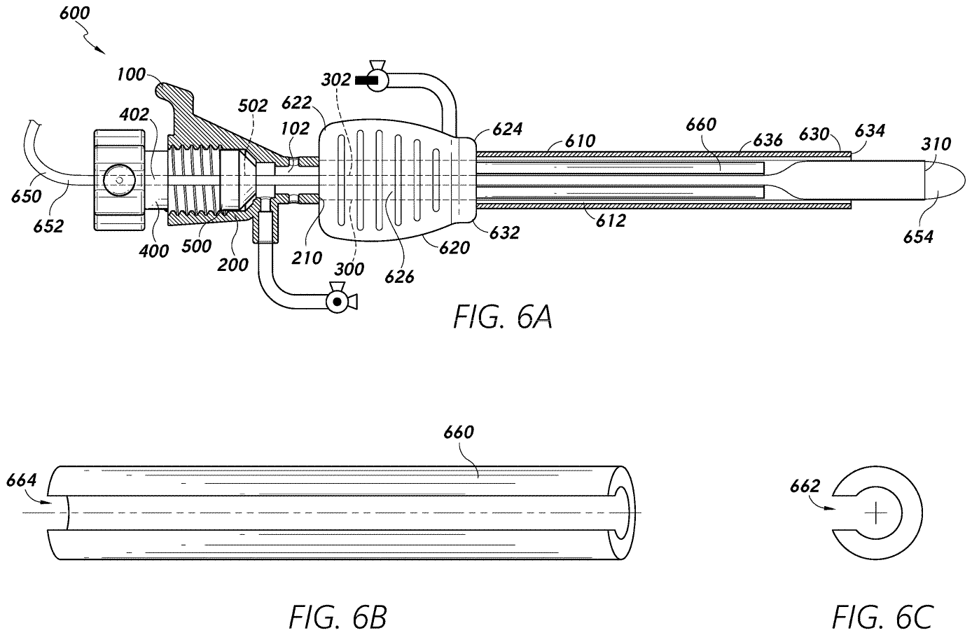

FIGS. 6A and 6B show an example of a delivery system 600 comprising the removable sheath assembly 100 and an outer sheath 610. Referring to FIG. 6A, the removable sheath assembly 100 can be used in combination with the outer sheath 610 to deliver a medical device 650 to a target site. In some instances, the medical device 650 can be pre-loaded within the removable sheath assembly 100. For example, the medical device 650 can be at least partially positioned within the delivery lumen 102 of the removable sheath assembly 100. In some instances, the medical device 650 can be pre-loaded such that a distal portion 654 of the medical device 650 extends distally of the removable shaft 300. A proximal portion 652 of the medical device 650 can extend proximally of the removable knob 400. As described herein, the delivery lumen 102 of the removable sheath assembly 100 can comprise the lumen 402 of the removable knob 400, the lumen 502 of the removable seal 500, and the lumen 302 of the removable shaft 300. The medical device 650 can be pre-loaded such that a respective portion of the medical device 650 is positioned within each of the lumen 402 of the removable knob 400, the lumen 502 of the removable seal 500, and the lumen 302 of the removable shaft 300.

The outer sheath 610 can comprise an outer sheath hub 620 and a sheath shaft 630. The sheath shaft 630 can extend distally from the distal end 624 of the sheath hub 620. The sheath shaft 630 can comprise a proximal end 632 associated with, such as coupled to, a distal end 624 of the sheath hub 620. The sheath shaft 630 can extend distally to a distal end 624 from the sheath hub 620. In some instances, the sheath shaft 630 can be flexible and/or pliable. The removable shaft 300 can be at least partially positioned within the delivery lumen 612 of the outer sheath 610. For example, a corresponding portion of the removable shaft 300 can be positioned within each of the lumen 626 of the outer sheath hub 620 and the lumen 636 of the outer sheath shaft 630. In some instances, the sheath shaft 630 can be an expandable shaft, including a reversibly expandable shaft. For example, corresponding portions of the sheath shaft 630 can expand along one or more lateral dimensions perpendicular or substantially perpendicular to a longitudinal axis of the sheath shaft 630. In some instances, corresponding portions of the sheath shaft 630 can expand radially as the removable shaft 300 is advanced through the lumen 636 of the outer sheath shaft 630 and can contract after the removable shaft 300 is withdrawn. Corresponding portions of the sheath shaft 630 can expand along one or more radially extending dimensions perpendicular or substantially perpendicular to the longitudinal axis of the sheath shaft 630 as the removable shaft 300 is advanced through the lumen 636. In some instances, the removable shaft 300 can be entirely positioned within the delivery lumen 612 of the outer sheath 610. In some instances, a distal end 210 of the removable hub 200 can be configured to be positioned against the proximal end 622 of the outer sheath hub 620 while the removable shaft 300 is positioned within outer sheath 610. A length of the removable shaft 300 extending distally of the removable hub 200 can be longer than a length of the outer sheath shaft 630 extending distally of the outer sheath hub 620. In some instances, a length of the removable shaft 300 extending distally of the removable hub 200 can be longer than a length of the outer sheath 610. For example, the distal end 310 of the removable shaft 300 can be distal of the distal end 634 of the outer sheath shaft 630. The longer length of the removable shaft 300 can be configured to provide protection for the medical device 650 as the medical device 650 is inserted through the outer sheath 610. The outer sheath 610 can be deformed due to curves and/or turns in the anatomical pathways through which the outer sheath 610 is positioned. Passing portions of the medical device 650 through the outer sheath 610 without a removable sheath assembly 100 can result in damage to the medical device 650, including bending of the medical device 650 and damage to one or more portions of an outer surface of the medical device 650. The removable sheath assembly 100 can provide additional mechanical strength, such as mechanical strength along a longitudinal axis of the medical device 650. The removable sheath assembly 100 can facilitate advancement of the desired portion of the medical device 650 through the outer sheath 610 while reducing or eliminating damage to the medical device 650.

In some instances, the medical device 650 can be a percutaneous ventricular assisted device (PVAD). The medical device 650 can be configured to be delivered to a target site within the heart. In some instances, at least a portion of the medical device 650 can be delivered into a chamber of the heart, including a chamber on the left side of the heart, including the left ventricle. For example, a distal portion 654 of the medical device 650 can be positioned into the left ventricle to facilitate blood flow out of the left ventricle. The medical device 650 can be used to pump blood from the left ventricle into the aorta.

In some instances, the delivery system 600 can optionally comprise a support member 660 configured to be positioned within the lumen 302 of the removable shaft 300. FIG. 6B shows the support member 660 in further detail. The support member 660 can be configured to provide additional mechanical strength along the longitudinal axis of the removable shaft 300. The support member 660 can be positioned partially around a portion of the medical device 650 extending through the lumen 302 of the removable shaft 300. The support member 660 can comprise a lumen 664 through which the portion of the medical device 650 can be positioned. In some instances, the support member 660 can assume a tubular shape having an opening 662 extending along its entire length. The opening 662 can facilitate the removal of the support member 660 from around the medical device. For example, as shown in FIG. 6C, a lateral cross section of the support member 660 along a dimension perpendicular or substantially perpendicular to its longitudinal axis can have a “C” shape. The support member 660 can be slid onto the medical device 650 through the opening 662 such that a corresponding portion of the medical device 650 is received by the lumen 664.

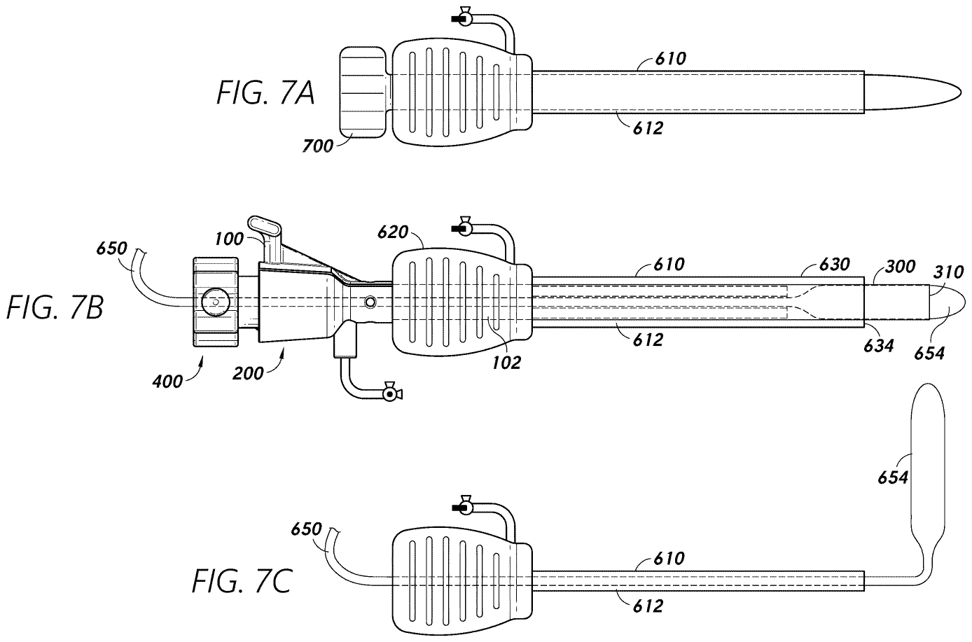

FIGS. 7A through 7C show an example of a process which uses the delivery system 600 described with reference to FIG. 6 to facilitate delivery of the medical device 650 to a target site. Referring to FIG. 7A, the outer sheath 610 can be positioned into a vessel, lumen, chamber, and/or cavity of the body. A dilator 700 can be positioned through the delivery lumen 612 of the outer sheath 610 to facilitate navigation of the outer sheath 610 through any tortuous anatomical pathways. After the outer sheath 610 is positioned within the desired vessel, lumen, chamber, and/or cavity, the dilator 700 can be withdrawn. Referring to FIG. 7B, the sheath assembly 100 can be subsequently inserted into the delivery lumen 612 of the outer sheath 610. For example, after the dilator 700 is withdrawn, at least a portion of the removable shaft 300 of the removable sheath assembly 100 can be subsequently inserted into the delivery lumen 612 of the outer sheath 610. As described herein, the removable shaft 300 can be advanced through the delivery lumen 612 of the outer sheath 610 such that the distal end 310 of the removable shaft 300 is distal of the distal end 634 of the outer sheath shaft 630. The medical device 650 can be pre-loaded within the removable sheath assembly 100 prior to positioning the removable shaft 300 into the delivery lumen 612 of the outer sheath 610. For example, the distal portion 654 of the medical device 650 can be distal of the distal end 310 of the removable shaft 300. The removable sheath assembly 100 comprising the medical device 650 pre-loaded therein can be positioned within the delivery lumen 612 of the outer sheath 610.

After the removable sheath assembly 100 is positioned at a desired position relative to the outer sheath 610, the medical device 650 can be advanced distally relative to the removable sheath assembly 100 and the outer sheath 610. In some instances, the medical device 650 can be advanced distally until the distal portion 654 of the medical device 650 is at a target site. Referring to FIG. 7C, the removable sheath assembly 100 can be removed after the medical device 650 has been advanced distally. For example, after a desired portion of the medical device 650 has been advanced distally of the distal end 310 of the removable shaft 300, the removable sheath assembly 100 can be removed, leaving the medical device 650 and the outer sheath 610 in place. The removable sheath assembly 100 can be removed from around the portion of the medical device 650 extending through its delivery lumen 102. For example, removing the removable sheath assembly 100 can comprise breaking and/or peeling away the removable hub 200, the removable knob 400, the removable seal 500 (not shown), and the removable shaft 300. As described herein, in some instances, the outer sheath shaft 630 can be expandable, including reversibly expandable. The outer sheath shaft 630 can contract after removal of the removable shaft 300, for example assuming a state having a smaller diameter than while the removable shaft 300 is extending therethrough. The outer sheath shaft 630 can contract and assume a size having a diameter which conforms to that of the portion of the medical device 650 extending therethrough.

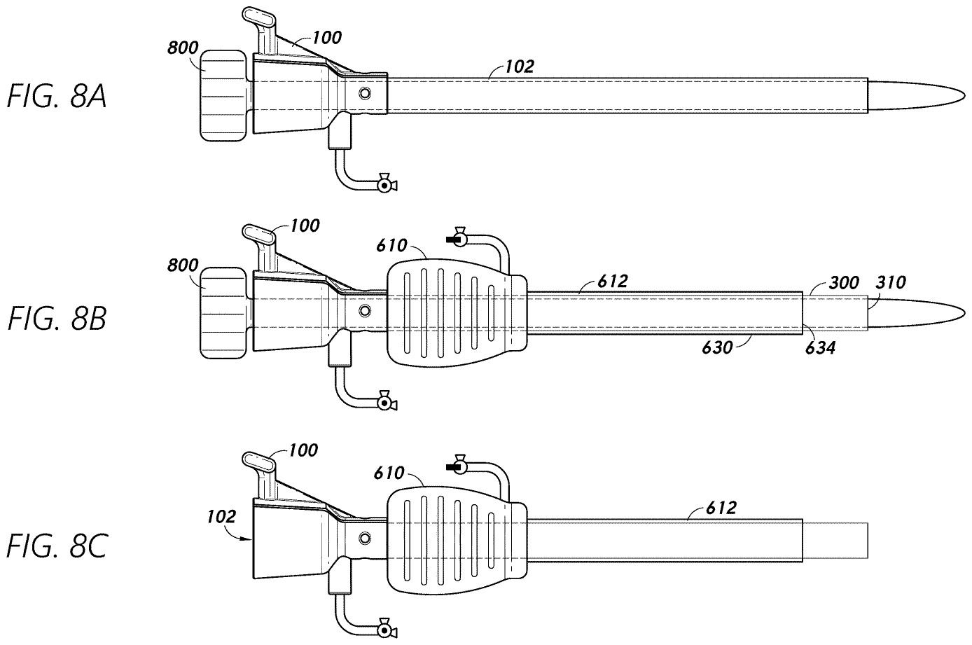

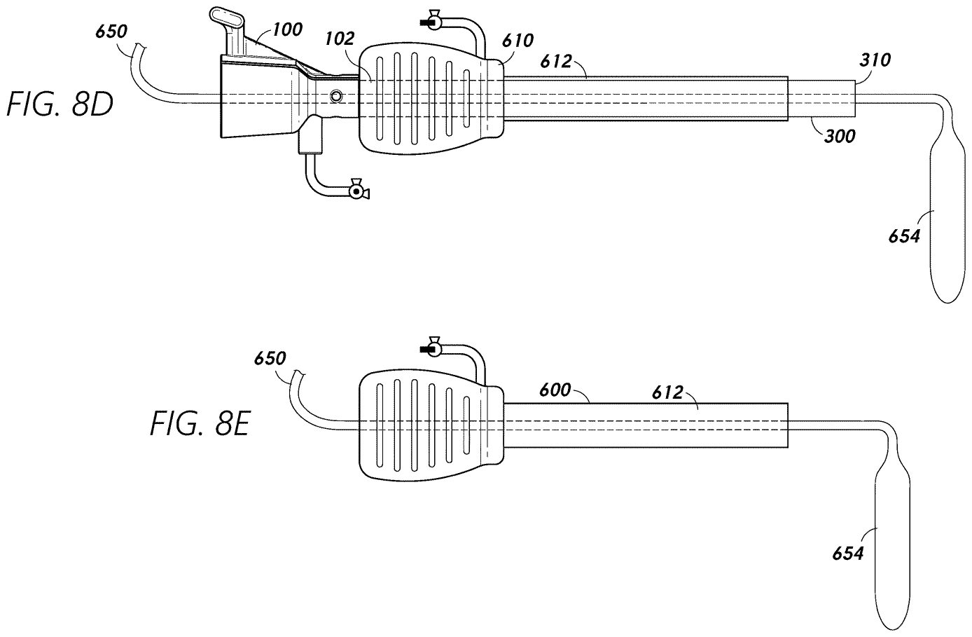

FIGS. 8A through 8E show another example of a process which uses the delivery system 600 described with reference to FIG. 6 to facilitate delivery of the medical device 650 to a target site. In the process described with reference to FIGS. 8A through 8E, the medical device 650 is not pre-loaded into the removable sheath assembly 100.

The process for delivering the medical device 650 can comprise positioning the outer sheath 610 into a desired vessel, lumen, chamber, and/or cavity of the body. As described herein, a dilator can be used with the outer sheath 610 to facilitate navigation of the outer sheath 610 through tortuous anatomical pathways. The removable sheath assembly 100 can be advanced through the outer sheath 610 after the outer sheath 610 has been positioned at a desired location within the vessel, lumen, chamber, and/or cavity. Referring to FIG. 8A, a second dilator 800 can be positioned through the delivery lumen 102 of the removable sheath assembly 100. Positioning the second dilator 800 through the delivery lumen 102 of the removable sheath assembly 100 can facilitate navigation of the removable sheath assembly 100 through the delivery lumen 612 of the outer sheath 610. Referring to FIG. 8B, the removable sheath assembly 100 comprising the second dilator 800 extending therethrough can then be positioned into the outer sheath 610. The removable sheath assembly 100 can be positioned into the outer sheath 610 as described herein. For example, at least a portion of the removable shaft 300 can be positioned within the delivery lumen 612 of the outer sheath 610 such that the distal end 310 of the removable shaft 300 is distal of the distal end 634 of the outer sheath shaft 630. The removable shaft 300 comprising a corresponding portion of the dilator 800 extending therethrough can be advanced through the delivery lumen 612 of the outer sheath 610 until the distal end 310 of the removable shaft 300 is at a desired position distal of the distal end 634 of the outer sheath shaft 630.

FIG. 8C shows the removable sheath assembly 100 positioned into the outer sheath 610, where the second dilator 800 has been removed. After a desired portion of the removable sheath assembly 100 is advanced through the delivery lumen 612 of the outer sheath 610, the second dilator 800 can be withdrawn from the delivery lumen 102. Referring to FIG. 8D, the medical device 650 can be subsequently advanced through the delivery lumen 102 of the removable sheath assembly 100. After the removable sheath assembly 100 is at a target position relative to the outer sheath 610, at least a portion of the medical device 650 can be advanced through the delivery lumen 102 of the removable sheath assembly 100 to deliver the distal portion 654 of the medical device 650 to the target site. After a desired portion of the medical device 650 has been advanced distally of the distal end 310 of the removable shaft 300, the removable sheath assembly 100 can be removed. The removable sheath assembly 100 can be removed from around the portion of the medical device 650 extending through the delivery lumen 102 of the removable sheath assembly 100. FIG. 8E shows that the medical device 650 positioned through the delivery lumen 612 of the outer sheath 610 without the removable sheath assembly 100.

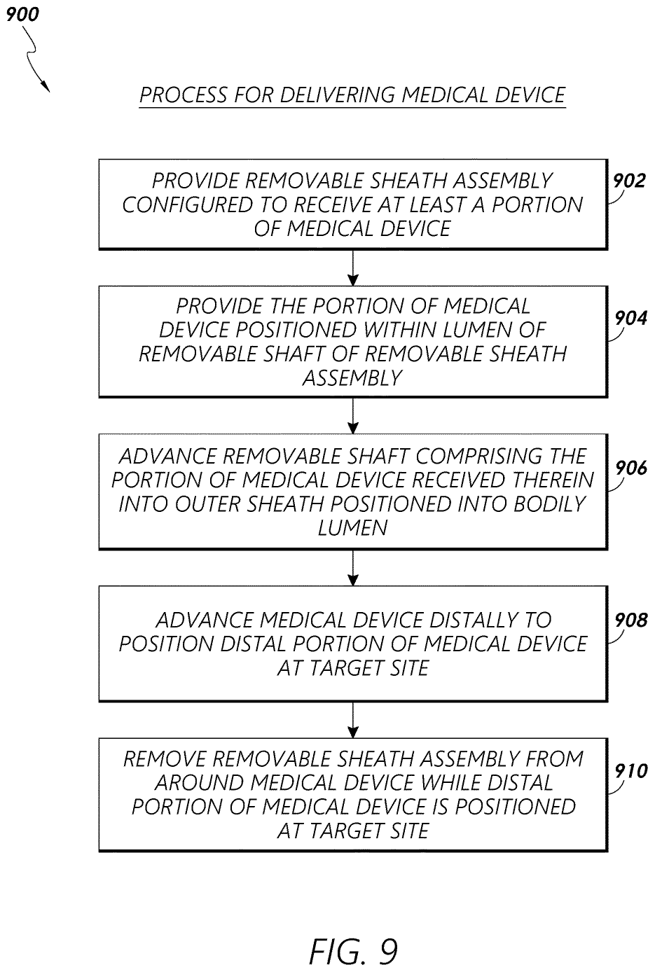

FIG. 9 is a process flow diagram of an example of a deployment process 900 for delivering a medical device to a target site using a removable sheath assembly comprising one or more features as described herein. In block 902, the process can involve providing a removable sheath assembly configured to receive at least a portion of a medical device. In some instances, the removable sheath assembly can comprise a removable hub configured to be separated into two circumferential hub portions spanning unequally sized circumferential segments around a longitudinal axis of the removable hub. A removable shaft can be associated with, such as coupled to, a distal portion of the removable hub such that separation of the circumferential hub portions from one another can be configured to separate the removable shaft into two circumferential shaft portions spanning unequally sized circumferential segments about a longitudinal axis of the removable shaft. The removable sheath assembly can comprise a removable knob being configured to be split into two halves and comprising a distal portion configured to be received within a lumen of the removable hub, the removable knob comprising a lumen configured to be aligned with a lumen of the removable shaft to allow advancement therethrough of at least a portion of the medical device. For example, the removable sheath assembly can comprise a delivery lumen comprising the lumen of the removable knob and the lumen of the removable shaft. At least a portion of the medical device can be advanced through the delivery lumen to facilitate delivery of the medical device to the target site.

In block 904, the process can involve providing the at least a portion of the medical device positioned within the lumen of the removable shaft. In some instances, the medical device can be positioned within the lumen of the removable shaft so as to provide desired protection for the medical device as it is advanced through tortuous anatomical pathways. For example, at least a portion of a distal portion of the medical device can be distal of a distal end of the removable shaft. In some alternative instances, a distal end of the medical device can be proximal of or aligned with the distal end of the removable shaft.

In block 906, the process can involve positioning the removable shaft comprising at least a portion of the medical device received therein into an outer sheath positioned into a bodily lumen, vessel, chamber, and/or cavity. As described herein, the medical device can be protected by the removable shaft as the medical device is navigated through the outer sheath positioned into the bodily lumen, vessel, chamber, and/or cavity.

In block 908, the process can involve advancing the medical device distally to position a distal portion of the medical device at a target site. As described herein, in some instances, the medical device can comprise a percutaneous ventricular assisted device (PVAD). In some instances, the medical device is configured to be delivered to a target site within a heart. For example, the distal portion of the medical device can be positioned into a heart chamber, including a heart ventricle. In some instances, the distal portion of the medical device can be positioned into a chamber on the left side of the heart, including a left ventricle.

In block 910, the process can involve removing the removable sheath assembly from around the medical device while the distal portion of the medical device is positioned at the target site. Removing the removable sheath assembly can comprise peeling and/or breaking away the removable sheath assembly from around the medical device. In some instances, a laterally extending handle of the removable hub can be distally pivoted to separate a first circumferential hub portion from a second circumferential hub portion. For example, the first circumferential hub portion can be lifted away and/or peeled away from the second circumferential hub portion, and removed from around the medical device. The second circumferential hub portion can then also be removed from around the medical device. The first circumferential hub portion can span a circumferential segment smaller than that of the second circumferential hub portion. A corresponding portion of the medical device can remain extending through the second circumferential hub portion after lifting and/or peeling away the first circumferential hub portion. For example, the portion of the medical device extending through the removable hub can be passed through the opening extending along the length of the second circumferential hub portion as a result of peeling away the first circumferential hub portion.

In some instances, a first laterally extending handle and a second laterally extending handle of the removable knob can be distally pivoted to effect splitting of the removable knob into the two halves. The two halves can then be removed from around the medical device.

In some instances, the removable sheath assembly can comprise a removable seal. The removable seal can be positioned within the lumen of the removable hub distally of the distal portion of the removable knob. The distal portion of the removable knob can be advanced distally within the lumen of the removable hub to effect longitudinal compression of the removable seal. The longitudinal compression can be configured to effect radial expansion of the removable seal, to thereby cause contact between the removable seal and a portion of the medical device extending through a lumen of the removable seal. A hemostatic seal can thereby be created around the portion of the medical device. Removing the removable sheath assembly from around the medical device can comprise removing the removable seal from around the medical device. Removing the removable seal can comprise passing a portion of the medical device extending through the lumen of the removable seal through a slit extending along a length of the removable seal.

In some instances, the removable knob can be twisted around a longitudinal axis relative to the removable hub to distally advance the removable knob relative to the removable hub and effect the longitudinal compression of the removable seal. In some instances, the removable knob can be distally translated relative to the removable hub to distally advance the removable knob relative to the removable hub and effect the longitudinal compression of the removable seal. For example, the removable knob can be pushed relative to the removable hub.

In some instances, the removable sheath assembly can be inserted into a femoral artery for delivery of at least a portion of the medical device into the heart. In some instances, the removable shaft of the removable sheath assembly can be positioned within the femoral artery. At least a portion of the medical device can be advanced through the delivery lumen of the removable sheath assembly. The distal portion of the medical device can be advanced from the femoral artery into the aorta. The medical device can be advanced such that the distal portion of the medical device is advanced from the aorta into the heart, including a chamber of the left heart. As described herein, the target location can be within a chamber of the left heart, including for example the left ventricle.

In some instances, a delivery catheter can be used to facilitate delivery of the medical device. At least a portion of the medical device can be received within a lumen of a delivery catheter while the delivery catheter is advanced through the delivery lumen of the removable sheath assembly. For example, the removable shaft of the removable sheath assembly can be positioned within the femoral artery. The delivery catheter carrying the medical device can be advanced from the femoral artery into the aorta. In some instances, a distal portion of the delivery catheter can remain in the aorta. For example, the medical device can be deployed through an opening at a distal end of the delivery catheter after the delivery catheter is desirably positioned. The distal portion of the medical device can be advanced into left heart, including the left ventricle. Alternatively, the distal portion of the delivery catheter can be positioned into the heart. The medical device can be deployed from the delivery catheter after the distal portion of the delivery catheter is desirably positioned within the heart.

### Additional Examples

Example 1: A sheath assembly comprising:
a removable hub having a lumen extending therethrough and comprising:
   a first circumferential hub portion that spans a first circumferential segment about a longitudinal axis of the removable hub,
   a second circumferential hub portion that spans a second circumferential segment about the longitudinal axis of the removable hub, the second circumferential segment being greater than the first circumferential segment, and
   first and second connector portions connecting the first circumferential hub portion and the second circumferential hub portion; and
a removable shaft configured to extend distally from a distal end of the removable hub, the removable shaft being configured such that separation of the first circumferential hub portion from the second circumferential hub portion causes a first circumferential shaft portion to separate from a second circumferential shaft portion.

Example 2: The assembly of any example herein, in particular example 1, further comprising:
a removable knob having a lumen extending therethrough, a distal portion of the removable knob being configured to be received within the lumen of the removable hub; and
a removable seal configured to be received within the lumen of the removable hub distally of the distal portion of the removable knob, the removable seal comprising a lumen extending therethrough and being configured to be aligned with the lumen of the removable shaft and the lumen of the removable knob, wherein advancing the removable knob distally within the lumen of the removable hub compresses the removable seal to thereby provide contact between the removable seal and a portion of a medical device extending through the lumen of the removable seal and form a hemostatic seal around the portion of the medical device.

Example 3: The assembly of any example herein, in particular example 2, wherein the removable knob is configured to be split into a first circumferential knob portion and a second circumferential knob portion, the first circumferential knob portion spanning a first circumferential segment about a longitudinal axis of the circumferential knob and the second circumferential knob portion spanning a second circumferential segment about a longitudinal axis of the circumferential knob, the first and second circumferential segments being equal.

Example 4: The assembly of any example herein, in particular example 3, wherein the removable knob comprises a first knob connector portion and a second knob connector portion extending along opposing portions of the removable knob, the first and second knob connector portions defining the first circumferential knob portion and the second circumferential knob portion.

Example 5: The assembly of any example herein, in particular examples 2 to 4, wherein a distal portion of the removable seal forms a frustoconical shape, and an inner wall surface portion of the removable hub defining the lumen of the removable hub forms a frustoconical shape configured to contact the frustoconically shaped portion of the removable seal such that advancing the removable knob distally presses the frustoconically shaped portion of the removable seal against the inner wall surface portion forming the frustoconical shape.

Example 6: The assembly of any example herein, in particular examples 2 to 5, wherein the removable seal comprises a slit extending along a length thereof to allow removal of the seal from around the medical device.

Example 7: The assembly of any example herein, in particular examples 2 to 6, wherein the removable knob comprises a plurality of grooves on an exterior surface of the distal portion, and the removable hub comprises a plurality of grooves on a proximal portion of an inner wall surface defining the lumen of the removable hub, the plurality of grooves of the removable hub being configured to mate with the plurality of grooves of the removable knob to allow controlled distal advancement of the removable knob within the lumen of the removable hub.

Example 8: The assembly of any example herein, in particular example 7, wherein the plurality of grooves of the removable knob comprise threads and the plurality of grooves of the removable hub comprise threads configured to mate with the threads of the removable knob, and the removable knob is configured to be rotated relative to the removable hub around a longitudinal axis of the removable knob to advance the removable knob distally within the lumen of the removable hub.

Example 9: The assembly of any example herein, in particular example 7 or 8, wherein the first connector portion comprise a first plurality of openings extending through a thickness of the first connector portion and the second connector portion comprises a second plurality of openings extending through a thickness of the second connector portion, the first and second plurality of openings positioned at trough portions of the corresponding plurality of grooves.

Example 10: The assembly of any example herein, in particular examples 2 to 9, wherein the removable knob comprises a first and a second handle in opposing orientation relative to one another and extending laterally from a proximal portion of the removable knob, and wherein pivoting the first and second handles distally is configured to effect separation of the removable knob into a first circumferential knob portion and a second circumferential knob portion.

Example 11: The assembly of any example herein, in particular examples 1 to 10, wherein the removable hub comprises a handle extending laterally from a proximal portion of the first circumferential hub portion, wherein pivoting the handle distally is configured to effect separation of the first circumferential hub portion from the second circumferential hub portion.

Example 12: The assembly of any example herein, in particular examples 1 to 11, wherein a proximal portion of the removable shaft is received within a distal portion of the lumen of the removable hub and coupled to the removable hub within the distal portion of the lumen.

Example 13: The assembly of any example herein, in particular examples 1 to 12, wherein the assembly is sterilized.

Example 14: A sheath assembly comprising:
a removable hub configured to separate into a first circumferential hub portion and a second circumferential hub portion, the second circumferential hub portion spanning a circumferential segment about a longitudinal axis of the removable hub larger than that of the first circumferential hub portion;
a removable shaft associated with a distal portion of the removable hub, and separation of the first circumferential hub portion from the second circumferential hub portion being configured to separate a first circumferential shaft portion associated with the first circumferential hub portion from a second circumferential shaft portion associated with the second circumferential hub portion, the second circumferential shaft portion spanning a circumferential segment about a longitudinal axis of the removable shaft larger than that of the first circumferential shaft portion; and
a removable knob, the removable knob being configured to be split into two halves, a distal portion of the removable hub being configured to be received within a proximal portion of a lumen of the removable hub, the removable knob having a lumen extending therethrough, the lumen of the removable knob being configured to be aligned with a lumen of the removable shaft to allow advancement of at least a portion of a medical device through the lumen of the removable knob and into the lumen of the removable shaft.

Example 15: The assembly of any example herein, in particular example 14, further comprising a removable seal comprising a lumen configured to allow positioning therethrough of a portion of the medical device, the removable seal being configured to be received within the lumen of the removable hub distally of distal portion the removable knob, and the removable seal being configured to be compressed by the removable knob to effect contact between the removable seal and a portion of the medical device extending through the lumen of the removable seal to form a hemostatic seal around the portion of the medical device.

Example 16: The assembly of any example herein, in particular example 15, wherein a distal end of the removable knob is configured to contact a proximal end of the removable seal to effect compression of the removable seal.

Example 17: The assembly of any example herein, in particular example 16, wherein a distal end of the removable seal is configured to be in contact with an inner wall surface defining the lumen of the removable hub.

Example 18: The assembly of any example herein, in particular examples 14 to 17, wherein the removable hub further comprises:
a first connector portion having a first plurality of openings extending through a thickness of the first connector portion;
a second connector portion having a second plurality of openings extending through a thickness of the second connector portion; and
a plurality of grooves on an inner wall surface defining the lumen of the removable hub, wherein the first and second plurality of openings are positioned at trough portions of the plurality of grooves.

Example 19: A sheath assembly comprising:
a removable hub comprising a first circumferential hub portion and a second circumferential hub portion being configured to be separated from one another, the first and second circumferential hub portions spanning a first and second circumferential segment about a longitudinal axis of the removable hub, respectively, the second circumferential segment being larger than the first circumferential segment;
a removable shaft associated with a distal portion of the removable hub, separating the first circumferential hub portion from the second circumferential hub portion being configured to separate a first circumferential shaft portion from a second circumferential shaft portion, the second circumferential shaft portion spanning a circumferential segment about a longitudinal axis of the removable shaft larger than that of the first circumferential shaft portion;
a removable knob comprising a distal portion configured to be received within a lumen of the removable hub; and
a removable seal configured to be received within the lumen of the removable hub and being positioned distally of the distal portion of the removable knob, the removable seal comprising a lumen extending therethrough, the lumen of the removable seal being configured to be aligned with a lumen of the removable knob and a lumen of the removable shaft to allow positioning therethrough of at least a portion of a medical device, and the removable knob being configured to be advanced distally within the lumen of the removable hub to exert a compressive force on the removable seal to radially expand the removable seal and effect contact between the removable seal and a portion of the medical device positioned through the lumen of the removable seal to provide a hemostatic seal around the portion of the medical device.

Example 20: The assembly of any example herein, in particular example 19, wherein a distal portion of the removable seal comprises a tapered configuration and wherein an inner wall surface of the removable hub defining the lumen of the removable hub comprises a tapered configuration configured to engage with the distal portion of the removable seal.

Example 21: The assembly of any example herein, in particular example 20, wherein the distal portion of the removable seal assumes a frustoconical shape, and wherein the inner wall surface of the removable hub defining the lumen of the removable hub forms a frustoconical shape configured to engage with the distal portion of the removable seal.

Example 22: The assembly of any example herein, in particular examples 19 to 21, wherein the distal portion of the removable knob comprises a plurality of grooves configured to mate with a plurality of grooves on an inner wall surface of the removable hub defining the lumen of the removable knob to allow turning of the removable knob relative to the removable hub to distally advance the removable knob relative to the removable hub.

Example 23: The assembly of any example herein, in particular examples 19 to 22, wherein the removable knob comprises a first connector portion and a second connector portion extending along opposing portions of the removable knob, the first and second connector portions being configured to allow separating the removable knob into two portions spanning equal circumferential segments about a longitudinal axis of the removable knob.

Example 24: A method of delivering a medical device, the method comprising:
providing a removable sheath assembly configured to receive at least a portion of a medical device, the removable sheath assembly comprising:
   a removable hub configured to be separated into two circumferential hub portions spanning unequally sized circumferential segments around a longitudinal axis of the removable hub,
   a removable shaft associated with a distal portion of the removable hub, separation of the circumferential hub portions from one another being configured to separate the removable shaft into two circumferential shaft portions spanning unequally sized circumferential segments about a longitudinal axis of the removable shaft, and
   a removable knob configured to be split into two halves, the removable knob comprising a distal portion configured to be received within a lumen of the removable hub, the removable knob comprising a lumen configured to be aligned with a lumen of the removable shaft to allow advancement therethrough of at least a portion of the medical device;
providing a portion of the medical device positioned within the lumen of the removable shaft;
advancing the removable shaft comprising the portion of the medical device received therein into an outer sheath positioned into a bodily lumen;
advancing the medical device distally to position a distal portion of the medical device at a target site; and
removing the removable sheath assembly from around the medical device while the distal portion of the medical device is positioned at the target site.

Example 25: The method of any example herein, in particular example 24, further comprising advancing the distal portion of the removable knob distally within the lumen of the removable hub to effect longitudinal compression of a removable seal positioned within the lumen of the removable hub and distally of the distal portion of the removable knob, the longitudinal compression of the removable seal being configured to effect radial expansion of the removable seal to contact a portion of the medical device extending through a lumen of the removable seal and create a hemostatic seal around the portion of the medical device.

Example 26: The method of any example herein, in particular example 25, wherein removing the removable sheath assembly comprises passing the medical device through a slit extending along a length of the removable seal to remove the removable seal from around the medical device.

Example 27: The method of any example herein, in particular example 25 or 26, further comprising twisting the removable knob around a longitudinal axis relative to the removable hub to distally advance the removable knob relative to the removable hub and effect the longitudinal compression of the removable seal.

Example 28: The method of any example herein, in particular example 25 or 26, further comprising distally displacing the removable knob relative to the removable hub to distally advance the removable knob relative to the removable hub and effect the longitudinal compression of the removable seal.

Example 29: The method of any example herein, in particular examples 24 to 28, wherein removing the removable sheath assembly comprises distally pivoting a laterally extending handle of the removable hub to separate a first circumferential hub portion from a second circumferential hub portion, the second circumferential hub portion spanning a larger circumferential segment about the longitudinal axis of the removable hub than that of the first circumferential hub portion.

Example 30: The method of any example herein, in particular examples 24 to 29, wherein removing the removable sheath assembly comprises distally pivoting a first laterally extending handle and a second laterally extending handle of the removable knob to effect splitting of the removable knob into the two halves.

Example 31: The method of any example herein, in particular examples 24 to 30, wherein the medical device is configured to be delivered to a target site within a heart.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of delivering a medical device, the method comprising:
providing a removable sheath assembly configured to receive at least a portion of a medical device, the removable sheath assembly comprising:
a removable hub configured to be separated into two circumferential hub portions spanning unequally sized circumferential segments around a longitudinal axis of the removable hub,
a removable shaft associated with a distal portion of the removable hub, separation of the circumferential hub portions from one another being configured to separate the removable shaft into two circumferential shaft portions spanning unequally sized circumferential segments about a longitudinal axis of the removable shaft; and
a removable knob configured to be split into two halves, the removable knob comprising a distal portion configured to be received within a lumen of the removable hub, the removable knob comprising a lumen configured to be aligned with a lumen of the removable shaft to allow advancement therethrough of at least a portion of the medical device;
providing a portion of the medical device positioned within the lumen of the removable shaft;
advancing the removable shaft comprising the portion of the medical device received therein into an outer sheath positioned into a bodily lumen;
advancing the medical device distally to position a distal portion of the medical device at a target site; and
removing the removable sheath assembly from around the medical device while the distal portion of the medical device is positioned at the target site.

2. The method of claim 1, wherein the removable sheath assembly further includes a removable seal positioned within the lumen of the removable hub and distally of the distal portion of the removable knob, the removable seal having a lumen.

3. The method of claim 2 further comprising advancing the distal portion of the removable knob distally within the lumen of the removable hub to effect longitudinal compression of the removable seal, the longitudinal compression of the removable seal being configured to effect radial expansion of the removable seal to contact a portion of the medical device extending through the lumen of the removable seal and to create a hemostatic seal around the portion of the medical device.

4. The method of claim 3, wherein the removable seal comprises a slit extending along a length thereof.

5. The method of claim 4, wherein removing the removable sheath assembly comprises passing the medical device through the slit of the removable seal to remove the removable seal from around the medical device.

6. The method of claim 3 further comprising twisting the removable knob around a longitudinal axis relative to the removable hub to distally advance the removable knob relative to the removable hub and effect the longitudinal compression of the removable seal.

7. The method of claim 3 further comprising distally displacing the removable knob relative to the removable hub to distally advance the removable knob relative to the removable hub and effect the longitudinal compression of the removable seal.

8. The method of claim 1, wherein removing the removable sheath assembly comprises distally pivoting a laterally extending handle of the removable hub to separate a first circumferential hub portion from a second circumferential hub portion, the second circumferential hub portion spanning a larger circumferential segment about the longitudinal axis of the removable hub than that of the first circumferential hub portion.

9. The method of claim 1, wherein removing the removable sheath assembly comprises distally pivoting a first laterally extending handle and a second laterally extending handle of the removable knob to effect splitting of the removable knob into the two halves.

10. The method of claim 1, wherein the medical device is configured to be delivered to a target site within a heart.

11. A method of delivering a medical device, the method comprising:
positioning an outer sheath into a bodily lumen;
inserting a portion of a removable sheath assembly within at least a portion of the outer sheath, the removable sheath assembly configured to receive at least a portion of a medical device, the removable sheath assembly comprising:
a removable hub forming a lumen and configured to be separated into two circumferential hub portions,
a removable shaft associated with a distal portion of the removable hub, the removable shaft forming a lumen and configured to be separated into two circumferential shaft portions,
a removable knob forming a lumen and configured to be split into two halves, and
a removable seal forming a lumen and a slit along a length thereof;
inserting a portion of the medical device through the lumen of the removable shaft;
advancing the medical device distally to position a distal portion of the medical device at a target site; and
removing the removable sheath assembly from around the medical device while the distal portion of the medical device is positioned at the target site.

12. The method of claim 11, wherein the two circumferential hub portions of the removable hub span unequally sized circumferential segments around a longitudinal axis of the removable hub.

13. The method of claim 12, wherein the two circumferential shaft portions span unequally sized circumferential segments about a longitudinal axis of the removable shaft.

14. The method of claim 13, wherein the two circumferential hub portions are connected to the two circumferential shaft portions.

15. The method of claim 11, wherein removing the removable sheath assembly includes separating the two circumferential shaft portions which causes separation of the two circumferential hub portions.

16. The method of claim 11, wherein the removable knob comprises a distal portion configured to be received within the lumen of the removable hub, the lumen of the removable knob configured to be aligned with the lumen of the removable shaft to allow advancement therethrough of at least a portion of the medical device.

17. The method of claim 11, wherein separation of the circumferential hub portions from one another is configured to separate the removable shaft into two circumferential shaft portions.

18. The method of claim 11 further comprising adhering a first circumferential shaft portion of the two circumferential shaft portions to a first circumferential hub portion of the two circumferential hub portions and a second circumferential shaft portion of the two circumferential shaft portions to a second circumferential hub portion of the two circumferential hub portions.

19. The method of claim 11, wherein removing the removable sheath assembly includes separating the two circumferential shaft portions which causes separation of the two circumferential hub portions due at least in part to adherence of the first circumferential shaft portion to the first circumferential hub portion and adherence of the second circumferential shaft portion to the second circumferential hub portion.

20. The method of claim 11, wherein removing the removable sheath assembly comprises passing the medical device through the slit of the removable seal to remove the removable seal from around the medical device.
